(19) United States
(12) Patent Application Publication
(10) Pub. No.: US 2005/0142232 A1
(43) Pub. Date: Jun. 30, 2005

(54) Novel herbal formulation as brain tonic

(75) Inventors: Palpu Pushpangadan, Uttar Pradesh (IN); Chandana Venkateswara Rao, Uttar Pradesh (IN); Ramaswami Kartik, Uttar Pradesh (IN); Shanta Mehrotra, Uttar Pradesh (IN); Raj Kumar Goel, Uttar Pradesh (IN); Satyabrata Acharya, Uttar Pradesh (IN); Madhavan Somanathan, Kerala (IN)

Correspondence Address: MERCHANT & GOULD PC, P.O. BOX 2903, MINNEAPOLIS, MN, 55402-0903, US

(73) Assignee: COUNCIL OF SCIENTIFIC AND INDUSTRIAL RESEARCH, New Delhi (IN)

(21) Appl. No.: 10/810,484

(22) Filed: Mar. 26, 2004

Related U.S. Application Data
(63) Continuation of application No. PCT/IB03/06206, filed on Dec. 26, 2003.

(51) Int. Cl. A61K 35/78

(52) U.S. Cl. 424/764; 424/776

(57) ABSTRACT

The invention provides a novel herbal formulation used to improve the memory and in treatment of amnesia as a brain tonic. Formulation(s) comprises of oleaginous oil of Sesamum indicum and the alcoholic extract of Centella asiatica. Conventionally used as emulsion or as a soft gelatin capsule for oral dosage forms. Sesamum indicum used in paralysis, aphrodisiac and dysmenorrhoea. The plant of Centella asiatica is considered as a useful alternative and tonic in diseases of the skin, nerves and blood.

### FIELD OF THE INVENTION

A novel synergistic herbal formulation as a brain tonic, cognition, improvement of memory and treatment of amnesia and in recalling of thoughts.

### BACKGROUND INFORMATION

A major discovery of the past two decades in the field of neurosciences has been the elucidation of behavioral, neurobiological and cellular basis of learning and memory processes. The brain is an assembly of interrelated neural systems that regulates their owns and each other's activity in a dynamic, complex fashion. Morphological properties of central neurons have been very useful for the description of the functional characteristics. Learning is defined as the acquisition of information and skills, and subsequent retention of that information is called memory. The subsequently deterioration of retention of information which in medical term is known as “amnesia”. Accordingly, effect of a wide variety of pharmacological agents or brain lesion on cognitive behavior have been studied and most validly interpreted as “enhancement or impairment” of learning and memory process. Learning and memory can be conceived as both psychological process as well as a change in synaptic neural connectivity. The development of scientifically validated models of ischemia induced-amnesia is vital to the analysis of the functional consequences of ischemic damage and to testing the behavioral efficacy of potentially therapeutic drugs. The role of medicinal plants in increasing the memory and acting as a brain tonic is still much underestimated. Besides this, certain oils have been found to be used as sedatives, central nervous system stimulants, adaptogens, bronchodilators, anti-stress and muscle relaxants (Singh et al, 2000). During late prenatal and early postnatal brain development, the cholinergic system in the central nervous system plays an important role in learning and memory function and that brain cholinergic hypofunction causes dementia with symptoms such as memory loss and disorientation in cerebrovascular or alzheimers disease (Coyle et al 1983). Following cerebral ischemia, a reduction in the cerebral blood flow and blood oxygen occur. It has also been reported that hypoxia induces a reduction of memory and judgement that is associated with a decrease in acetylcholine synthesis (Gibson and Duffy, 1981). Principally, main characteristic of memory formation in animals, as well as in human being, is its progression from a short-lived labile form to a long-lasting stable form. During this period of consolidation, memory can be disrupted by administration of a wide variety of amnesia-inducing agents. Electroconvulsive shock, hypothermia and hypoxia are non-invasive procedures that can render the animal unconscious, inducing retrograde amnesia through mechanisms correlated to the practical utility to the clinical drugs. The retrieval hypothesis postulates that amnesic agents disrupt memory recall rather than storage, as the effect of some agents diminish over time resulting in the reappearance of normal memory retention. The consolidation of information is mediated by limbic structures, with the hippocampal formation particularly playing a key role in memory processing. The major pathways have been proposed in the limbic system and cortical structures as being responsible for the neuronal interconnection of information processing. Drugs like amphetamine, caffeine-containing substances which has a stimulant activity on memory. Accordingly, studies shown that the herbal formulation(s) having the property of improving the memory and used in treatment of amnesia as a brain tonic and acting as a central antioxidant.

### OBJECT OF THE INVENTION

The main object of the present invention provides a synergistic herbal formulation as a brain tonic, cognition, recalling of thoughts and as an antioxiadant capable of treating or preventing amnesia and having property for improving memory.

Another object of the present invention provides a method of preparing a synergistic herbal formulation as a brain tonic, cognition, recalling of thoughts and as an antioxiadant capable of treating or preventing amnesia and having property for improving memory.

Yet another object of the present invention provides a use of synergistic herbal formulation as a brain tonic, cognition, recalling of thoughts and as an antioxiadant capable of treating or preventing amnesia and having property for improving memory.

### SUMMARY OF THE INVENTION

The present invention provides a herbal formulation useful in the treatment of herbal dosage form from the seed oil of Sesamum indicum used as a brain tonic and cognition. The herbal oil comprising of sesamin, sesamolin, sesamol (a phenolic antioxidant) vitamins, proteins and aminoacids. Sesame oil varies from light to deep reddish yellow in colour. It is used as nourishing food and flavoring agent. Sesamum seeds are considered as emollient, diuretic, lactagogue and a nourishing tonic and said to be useful in curing bleeding piles and also from the fresh leaves extract of centilla asiatica that is having a potential memory enhanching role and also we have found to produce transquilizing effects. The extracts comprising of centoic acid, centellic acid, oleic acid linolic acid, linolenic and lingocericacid. It is used as acures for leucoderma, bronchitis, kapha, enlargement of spleen (Ayurveda). It is also used as a cardio tonic diuretic and also used to improve appetite (Yunani). It was shown that it produce a significant improvement in general ability and behavioural pattern.

### DETAILED DESCRIPTION OF THE INVENTION

Sesamum indicum Linn. Family: Pedaliacae

Botanical description: A genus of annual or perennial herbs or occasionally shrubs found in the warmer regions of Africa, Asia and Australia. About six species are recorded in India of which Sesamum indicum is widely cultivated. An erect, branched or un branched annual 60-180 cm high, cultivated throughout the plains of India and upto an altitude of 1,200 m. Leaves 7.5-12.5 cm simple (or) when variable, with upper ones narrowly oblong, middle ones ovate and toothed and the lower ones lob ate or pedatisect. Flowers white, pink or mauve pink with darker markings, borne in racemes in the leaf axils, fruit capsular, oblong. Quadrangular, slightly compressed, deeply 4-grooved, 1.5-5 cm long, seeds black, brown or white 2.5-3 mm long and 1.5 mm broad. (Wealth of India, 1992)

Medicinal uses: seasame seed is used as a nourishing food and also as flavouring agent. It is invariably dehulled for use of food. The method of dehulling consists in soaking the seed in cold water overnight, followed by partial drying and rubbing against a rough surface. Sesamin and sesamolin exhibit little antioxidant activity. (Wealth of India, 1992)

Phytochemistry: The oleaginous edible seeds of Sesamum indicum esteemed for their oil, have acquired, in recent years, additional importance as a source of protein for human nutrition. It varies in colour from white, through brown, to black. Analyses of seeds grown in various parts of the world for their proximate composition gave values which lies within the ranges (in g/100 g, of dry seed): moisture, 4.1-6.5; ether extr., 43.0-56.8; protein 17.6-26.4; crude fibre, 2.9-8.6; carbohydrates, 9.1-25.3; it also contain vitamins, fairly rich in thiamine and niacin. It also contains other vitamins like riboflavin, nicotinic acid, 80.0; pantothenic acid, 9.5; and ascorbic acid in trace amount. It also contains carbohydrates from the alcoholic extraction of deffated seed meal (% dry-matter basis) like glucose, 2.6; sucrose, 0.57; galactose, 1.1; and raffinose in trace amount. I contain the principal protein globulin (alpha and beta globulin). Sesame oil is rich in oleic and linoleic acids, which together constitute account for 85 percent of total fatty acids. The main constituent of studying its minor constituents of the oil contain two constitutes, sesamin and sesamolin, which are not found in any vegetable oil and responsible for the synergistic effect on the action of insecticides. Another compound, sesamol, aphenolic antioxidant, is usually present in traces.

Pharmacology: The sesamum oil having the antioxidant activity. Sesamum seeds are considered emollient, diuretic, lactagogue and a nourishing tonic. They are said to be helpful in piles, a paste of seeds mixed with butter being used in bleeding piles. A decoction of seeds is said to be an emmenagogue and also use in cough. Combined with linseed, the decoction of seeds is used as an aphrodisiac. A plaster made of ground seeds are applied to burns, scalds, and etc. and a poultice of the seeds is applied to ulcers. Powdered seeds are used in amenorrhoea and dysmenorrhoea (Kirt, & Basu, II, 1859; Nadkarni, I, 1128).

Centella asiatica Family: Umbelliferae

Botanical description: A slender herbaceous creeping; stem long, prostate coming off from the leaf-axils of a vertical rootstock, filiform, often reddish, and with long internodes, rooting at the nodes. Leaves 1.3-6.3 cm in diameter., several from the rootstock which often have much elongated petioles, and 1-3 from each nodes of the stems, orbulicar, reniform, rather broader than long, more or less cupped, entire or shallowly crenate, glabrous on both sides, and with numerous slender nerves from a deeply cordate base; petioles very variable in length 7.5-15 cm long or more, channelled, glabrous or nearly so; stipule short, adnate to the petioles forming a sheating base. Flower in fasicicled umbel consisting of 34 pink, sessile (rarelt pedicelled) flowers;

peduncles pubescent or glabrous, short, pink bracts ovate, acute, concave, 2 beneath each umbel. Calyx-teeth 0. petals minute, pink, ovate acute. Fruit 4 mm. Long, longer than broad, ovoid, hard, with thickened pericarp, reticulate-rugose, often crowned by the persistent petals, the peimary and secondary ridges distinct. Distributed through India, Ceylon and also in tropical and subtropical region of the world.

Medicinal uses: The plant is Acrid, bitter, digestible, laxative, cooling effect, tonic, and antipyretic, improve appetite (Yunani), cures leucoderma anaemia, urinary discharge, disease of blood, use in insanity (Ayurveda). The plant has bad taste; soporific, sedatives to the nerves, acts as a cardiotonic clears the voice and the brain; cures hiccough, headache. The plant is considered as a useful alernative and tonic in diseases of skin, nerves. In some part of India, the people are in the habit of taking the powdered dried leaves with milk for improving their general intelligence the leaves are said to be useful in syphilitic skin diseases, both externally and internally; and on the malabar coast, the plant is one of the remedies for leprosy. It is also a popular remedy for slight dysenteric derangement of bowls to which children are subject: three or four leaves are given with cumin and sugar, and the pounded leaves are applied to navel. In konkan, one or two leaves are given every morning to cure stuttering; and the juice is applied (generally as a lep with Cadamba bark, and black cumin) to skin eruption supposed to arise from heat of blood.

Phytochemistry: The alcoholic extract of herb an essential oil, green in colour and possing the strong odour of the herb, fatty oil, sitosterol and a resinous substance have been obtained. The fatty oil consists of the glyceride, linolic, lignoceric, palmitic and stearic acid. An alkaloid hydrocortylin has been obtained from the dried plant. Vellarine, pectic acids are present in the leaves and roots. The plants also contain ascorbic acid in a conc. Of 13.8 mg %. A glycoside asiaticoside has been isolated from the plant. The major componant of the triterpine mixture is centoic acid.

Pharmacology: The ususal dose for the oral administration is 5-10 grains of the plant powder thrice daily. In larger doses, the drug is a simplifying narcotic, producing giddiness and some times coma. The alcoholic extract produce tranquillising effect in rats. It was found non-toxic up to a dose of 350-mg/kg i.p. The alcoholic and aqueous extracts antagonise spontaneous contraction and also caused relaxation of musculature of isolated ileum of rat. The alcoholic extract was found to have depressant effect in rat in toxic doses. The glycosidal fractions have a sedative action in rats. It decreases the tone and diminished the amplitude of contractions of isolated ileum of rabbit and albino rat. In anaesthetised dogs, it produces slight respiratory stimulation, hypotension and bradycardia. The alcoholic extract of entire plant was found to possess anti-protozoal activity against E. histolytica. (Wealth of India, 1992, 115-118; Kirtikar and Basu, Indian Medicinal Plant, Vol 5, 2001 p. 219). Accordingly, the main embodiment of the present invention relates to a synergistic herbal formulation as a brain tonic, cognition, recalling of thoughts and as an antioxiadant capable of treating or preventing amnesia and having property for improving memory, said formulation comprising pharmaceutically acceptable amounts of extracts from plants Centella asiatica and Sesamum indiucm optionally along with acceptable salt/s, carrier/s or dilutent/s.

Another embodiment of the present invention relates to a method of preparing a synergistic herbal formulation as a brain tonic, cognition, recalling of thoughts and as an antioxiadant capable of treating or preventing amnesia and having property for improving memory as a brain tonic and as an antioxiadant capable of treating or preventing amnesia and having property for improving memory, said method comprising steps of:
(a) extracting the powdered material obtained from seeds of Sesamum indicum and leaves of Centella asiatica in aqueous alcohol,
(b) filtering the extract of step (a) to remove the debris,
(c) concentrating and lyophislizing the filtrate obtained from step (b) at a temperature of less than about 55° C., and
(d) mixing the plant extracts obtained in step (c) with carbohydrates of about 70% and alcohol of about 12% to make a volume of 100 ml to obtain the formulation

Yet another embodiment of the present invention relates to the aqueous alcohol in steps (a) and (d), wherein the aqueous alcohol is ethanol.

One more embodiment of the present invention relates to the aqueous alcohol in step (a) wherein the aqueous alcohol is about 60%.

Another embodiment of the present invention relates to the aqueous alcohol in step (a) wherein the aqueous alcohol is about 50%.

One more embodiment of the present invention relates to the temperature wherein the temperature in the step (b) is about 50° C.

Yet another embodiment of the present invention relates to the carbohydrates, wherein the carbohydrates in step (d) are selected from sucrose or lactose.

In one more embodiment of the present invention relates to the carbohydrates, wherein carbohydrate concentration is about 66%.

Another embodiment of the present invention relates to the method of treating and or preventing amnesia and improving memory in mammals, particularly humans said method comprising administering synergistic herbal formulation of extracts from plants Centella asiatica and Seasmum indicum optionally along with pharamaceutically acceptable salt/s, carrier/s or dilutent/s to a subject.

Another embodiment of the present invention relates to Sesamum indicum oil and Centella asiatica oil wherein Sesamum indicum oil is in the range of about 2-20% and Centella asiatica oil is in the range of about 1-15%.

One more embodiment of the present invention relates to Sesamum indicum oil and Centella asiatica oil wherein Sesamum indicum oil is about 10% and Centella asiatica oil is 5%.

In another embodiment of the present invention relates to the extract of the formulation wherein the said formulation comprises Sesamum indicum oil is about 4% and Centella asiatica oil is about 2%.

Another embodiment of the present invention relates to the pharmaceutically acceptable dilutent/s, carrier/s, salt/s, wherein said pharmaceutically acceptable dilutent/s, carrier/s, salt/s are selected from group comprising of lactose, mannitol, sorbitol, microcrystalline cellulose, sucrose, sodium citrate, sodium chloride or dicalcium phosphate.

Still another embodiment of the present invention relates to the formulation, wherein the said formulation has a high antioxidant, cooling, oleaginous, diuretic and nerve relaxant properties.

Yet another embodiment of the present invention relates to the formulation wherein said formulation may be delivered in form of capsule, tablet, syrup, suspension, pills or elixirs.

Another embodiment of the present invention relates to the extract of the formulation wherein said extract of the formulation is obtained from leaves of Centella asiatica and seeds of Sesaumum indicum.

One more embodiment of the present invention relates to the plant parts, wherein plant parts are selected from a group consisting of seeds of white and black varieties and leaves.

In another embodiment of the present invention relates to the use of formulation wherein said formulation is used for curing migraine, vertigo, leucoderma, anaemia and improve appetite.

Yet another embodiment of the present invention relates to the formulation, wherein formulation may be used for curing wounds, fractures, syphilitic skin diseases, both externally and internally and also in treatment of leprosy and to ameliorate the symptoms of disease and to improve the general health of the patient.

Still another embodiment of the present invention relates to the formulation wherein the said formulation is used to reduce the pain of piles, stomachic, and enlargement of spleen.

Another embodiment of the present invention relates to the dosage of the formulation wherein said dosage of the formulation in the range of about 20-110 mg/kg does not show abnormality of the locomotor activity, on passive avoidance test showed significant and dose dependent activity, showed significant and dose dependent antioxidant activity of the frontal cortex and of striatum regions of the brain.

Another embodiment of the present invention relates to the dosage of the formulation wherein said dosage of the formulation in the range of about 25-100 mg/kg does not show abnormality of the locomotor activity, on passive avoidance test shows significant and dose dependent activity and shows significant and dose dependent antioxidant activity of the frontal cortex and of striatum regions of the brain.

Still another embodiment of the present invention relates to the formulation wherein formulation reduces the latency period in the range of about 0.05 to 2.0 seconds.

Still another embodiment of the present invention relates to the formulation wherein formulation reduces the latency period in the range of about 0.18 to 1.22 seconds.

Another embodiment of the present invention relates to the formulation wherein said formulation lowers the number of mistakes in the range of about 1 to 35.

Another embodiment of the present invention relates to the formulation wherein said formulation lowers the number of mistakes in the range of about 6.1 to 27.

One more embodiment of the present invention relates to the formulation, wherein the said formulation enhances the body weight in the range of about 140 to 170 gms.

One more embodiment of the present invention relates to the formulation, wherein the said formulation enhances the body weight in the range of about 141.6 to 168.7 gms.

Still another embodiment of the present invention relates to the formulation, wherein the said formulation enhances the kidney weight in the range of about 0.80 to 1.5 gms.

Still another embodiment of the present invention relates to the formulation, wherein said formulation enhances the kidney weight in the range of about 0.82 to 1.03 gms.

Yet another embodiment of the present invention relates to the formulation wherein said formulation enhances the liver weight in the range of about 4 to 7 gms.

Yet another embodiment of the present invention relates to the formulation wherein said formulation enhances the liver weight in the range of about 5.26 to 6.42 gms.

One more embodiment of the present invention relates to the formulation wherein said formulation enhances the spleen weight in the range of about 0.60 to 0.80 gms.

One more embodiment of the present invention relates to the formulation wherein said formulation enhances the spleen weight in the range of about 0.63 to 0.76 gms.

Another embodiment of the present invention relates to the formulation wherein said formulation under non-stress conditions lowers the lipid peroxidase (LPO) activity in the frontal cortex and stratium regions of the brain in the range of 1.0 to 5.0.

Another embodiment of the present invention relates to the formulation wherein said formulation under non-stress conditions lower the lipid peroxidase (LPO) activity in the frontal cortex and stratium regions of the brain in the range of 0.74 to 3.48.

Still another embodiment of the present invention relates to the formulation wherein said formulation under non-stress conditions enhances the catalase (CAT) activity in the frontal cortex and stratium regions of the brain in the range of 22 to 40.

Still another embodiment of the present invention relates to the formulation wherein the said formulation under non-stress conditions enhances the catalase (CAT) activity in the frontal cortex and stratium regions of the brain in the range of 24.5 to 35.3.

Another embodiment of the present invention relates to the formulation wherein said formulation under non-stress conditions enhances the superoxide dismutase (SOD) in the frontal cortex and stratium regions of the brain activity in the range of 22 to 40.

Another embodiment of the present invention relates to the formulation wherein said formulation non-stress conditions enhance the superoxide dismutase (SOD) activity in the frontal cortex and stratium regions of the brain in the range of 23.2 to 30.3.

Yet another embodiment of the present invention relates to the formulation wherein the said formulation under stress conditions lower the LPO activity in the frontal cortex and stratium regions of the brain in the range of about 1 to 7.

Yet another embodiment of the present invention relates to the formulation wherein the said formulation under stress conditions lower the LPO activity in the range of about 2.8 to 4.86.

One more embodiment of the present invention relates tot the formulation wherein the said formulation under chronic stress conditions enhance CAT activity in the frontal cortex and stratium regions of the brain in the range of 10 to 25.

One more embodiment of the present invention relates tot the formulation wherein the said formulation under chronic stress conditions enhance CAT activity in the frontal cortex and stratium regions of the brain in the range of 12.4 to 22.5.

Yet another embodiment of the present invention relates to the formulation wherein the said formulation under chronic stress conditions lower the SOD activity in the frontal cortex and stratium regions of the brain in the range of 20 to 35.

Yet another embodiment of the present invention relates to the formulation wherein the said formulation under chronic stress conditions lower SOD activity in the frontal cortex and stratium regions of the brain in the range of 21 to 33.

The following examples are given by way of illustration of the present invention and therefore should not be construed to limit the scope of the present invention.

### EXAMPLES

### Example 1

The invention is further illustrated by the following non-limiting examples.

Formulation (F1)

| | |
|---|---|
| Sesamum indicum | 2 wt. % |
| Sucrose/Lactose | 66.7 g/1.2 g |
| Alcohol | 10 wt. % |
| Water | q.s. to make 100 ml |

Formulation (F2)

| | |
|---|---|
| Centella asiatica | 2 wt. % |
| Sucrose/Lactose | 66.7 g/1.2 g |
| Alcohol | 10 wt. % |
| Water | q.s. to make 100 ml |

Formulation (F3)

| | |
|---|---|
| Sesamum indicum | 4 wt. % |
| Centella asiatica | 2 wt. % |
| Sucrose/Lactose | 66.7 g/1.2 g |
| Alcohol | 10 wt. % |
| Water | q.s. to make 100 ml |

Sesamum indicum, and Centella asiatica were collected and dried in shade. The dried material (1 Kg) is then powdered and extracted with 50% aqueous alcohol (3 L) for 5 days. At the end of this, the solvent is decanted and filtered if necessary to remove the plant debris. The extract is then concentrated under vacuum at less than 50° C. Then the extract is lyophilised to obtain the extract in powder form. Mix the plant extracts and dissolve them in 500 ml 10% alcohol, filter the solution and add specified quantity of sugar and heat the until the sugar dissolves and then cool and make up the volume with required amount of water to make 100 ml.

The formulation is useful to a brain tonic and cognition. Accordingly, the investigation deals with the oral dosage form has been described in detail giving the formula of the ingredients along with the method and mode of usage of the standardzed edible oil.

Locomotor Activity:

The locomotor activity was measured by an open-field method. The apparatus put in the soundproof, darkened room was a round open field (bottom diameter, 60 cm; height, 50 cm). The bottom was divided into 19 parts that were equal in area. A 100-W lamp was positioned 80 cm above the bottom each rat was placed at the center of the open field and the spontaneous activity (ambulation and rearing) was recorded for 5 min.

Passive Avoidance Task (Step-Down Test):

A step-down passive avoidance was examined using apparatus consisted of a box (25×25×40 cm), a floor with stainless-steel grid 2 mm in diameter at 8-mm intervals, and a rubber platform (4 cm diameter, 4 cm height) set on the grid in one corner. Electric stimulation was given through the grid connected with a scrambled shock generator. After 24 hr of cerebral ischemia/scopolamine (0.4 mg/kg, i.p.), an acquisition trail was performed. For this trial, each rat was placed gently on the platform and allowed to habituate freely for 3 min, and then electric shock (0.4 mA) were delivered to the grid. If the rat stepped down from the platform, the electric shock was delivered to the rat on the grid floor. The cut off time was 2 min. A retention trail was performed 24 hr after the acquisition trail. Each rat was again placed on the platform. The time (step-down latency) that elapsed until the rats stepped down from the electric grid of the platform to shock free zone was recorded. If the rat did not step down from the platform within 2 min's, the retention trail was terminated and the maximal step down latency of 2 min was recorded. An error was counted when ever the rat stepped down from the platform and the number of error made in 2 min was recorded (Tables 1 to 4).

Foot Shock-Induced Chronic Stress:

The rats were subjected to daily 1 hr footshock through a grid floor in a Perspex box for 21 days. The duration of each shock (2 mA) and the intervals between the shock was randomly programmed between 3-5 sec and 10-110 sec, respectively and brain tissue was separated for the detailed central antioxidant enzymes (Tables 5 to 6).

TABLE 1
Effect of formulation F1 on impairment of
memory acquisition in step-down test in mice

| | |
|---|---|
| Treatment | Memory parameters |

| (Mg/kg) | Latency (sec) | No of mistakes |
|---|---|---|
| Control | 3.81 ± 0.01 | 20.2 ± 3.5 |
| Scopalamine 0.4 | 7.7 ± 0.04^{c} | 66.8 ± 8.9^{c} |
| F1 25 | 7.2 ± 0.03^{y} | 45.3 ± 7.6 |
| F1 50 | 6.9 ± 0.03^{y} | 31.5 ± 3.4^{x} |
| F1 100 | 3.26 ± 0.02^{y} | 10.0 ± 2.9^{y} |

P: ^{c}<0.001 compared to control group.
P: ^{x}<0.01 and ^{y}<0.001 compared to scopolamine group.

Note: There is no mortality/gross abnormality was observed in the animals during the treatment of Sesamum indicum oil.

The formulation F1 contains the Sesamum oil only

The results of the table 1 represent a dose response decrease in the number of mistakes done by the animals. Whereas the scopolamine treated group showed a significant increase in the number of mistakes. Therefore latency period was increased with F1 formulation and showed a significant result.

TABLE 2
Effect of formulation F2 on impairment of
memory acquisition in step-down test in mice

| | |
|---|---|
| Treatment | Memory parameters |

| (Mg/kg) | Latency (sec) | No of mistakes |
|---|---|---|
| Control | 4.32 ± 0.02 | 21.2 ± 3.6 |
| Scopalamine 0.4 | 7.8 ± 0.03^{c} | 65.3 ± 8.6^{c} |
| F2 25 | 6.3 ± 0.02^{x} | 53.2 ± 7.1 |
| F2 50 | 5.9 ± 0.02^{x} | 46.2 ± 7.3^{x} |
| F2 100 | 4.2 ± 0.01^{x} | 32.1 ± 4.1^{y} |

P: ^{c}<0.001 compared to control group.
P: ^{x}<0.05 and ^{y}<0.01 compared to scopolamine group.

Note: There is no mortality/gross abnormality was observed in the animals during the treatment of without Sesamum indicum oil containing formulation.

The formulation F2 contains Centella asiatica only. The result showed a significant decrease in number of mistakes but when we see the table 1 the number of mistakes done with F1 formulation is less than F2 formulation. Whereas the scopolamine showed a significant increase in number of mistakes.

TABLE 3
Effect of formulation F3 on impairment of
memory acquisition in step-down test in mice

| | |
|---|---|
| Treatment | Memory parameters |

| (Mg/kg) | Latency (Sec) | No of mistakes |
|---|---|---|
| Control | 3.89 ± 0.02 | 20.9 ± 3.2 |
| Scopalamine 0.4 | 7.8 ± 0.04^{c} | 69.8 ± 8.9^{c} |
| F3 25 | 1.2 ± 0.02^{x} | 20.3 ± 6.9^{x} |
| F3 50 | 0.8 ± 0.03^{x} | 10.5 ± 3.4^{x} |
| F3 100 | 0.2 ± 0.02^{x} | 2.9 ± 3.2^{x} |
| Tacrine 1 | 0.1 ± 0.02^{x} | 2.4 ± 2.6^{x} |

P: ^{c}<0.001 compared to control group.
P: ^{x}<0.05 and ^{y}<0.01 compared to scopolamine group.

Note: There is no mortality/gross abnormality was observed in the animals during the treatment of Sesamum indicum oil containing formulation.

F3 formulation contains Sesamum indicum plus Centella asiatica.

The results of Table 3 represents a highly significant effect with the dose. Whereas Tacrine is a positive control showed a better result but as a synthetic drug the side effect on saturation of various receptors cannot be ignored. The scopolamine treated animals showed negative results of losing the memory and increased the number of mistakes. Therefore F3 formulation showed a synergetic effect than that of F1 (Table 1) and F2 (Table 2) formulations.

Tacrine (1,2,3,4-tetrahydro-5-aminoacridine or THA) (Summers et al, Clinical Tox 1980; 16(3): 269-281) is more effective in improving memory in Alzheimer's patients and used to treat the symptoms of Alzheimer's disease, but it does not cure the disease and it also upset the stomach, vomiting, diarrhea, heartburn, muscle aches, headache, loss of appetite etc.

TABLE 4
Effect of formulation (F3) on relative mean ± SEM
organ weights of rats (n = 6)

| Type of | Treatment | | | | |
|---|---|---|---|---|---|
| treatment | group | Body weight (g) | Kidney (g) | Liver (g) | Spleen (g) |
| 6 days | Control | 150.8 ± 10.1 | 0.93 ± 0.05 | 5.81 ± 0.44 | 0.64 ± 0.05 |
| oral | F3 25 | 154.2 ± 11.6 | 0.98 ± 0.05 | 5.85 ± 0.59 | 0.67 ± 0.04 |
| treatment | F3 50 | 152.5 ± 10.9 | 0.91 ± 0.09 | 5.97 ± 0.45 | 0.74 ± 0.2 |
| | F3 100 | 157.2 ± 11.5 | 0.97 ± 0.07 | 5.88 ± 0.62 | 0.68 ± 0.04 |

F3 formulation contains mixure of Sesamum indicum and Centella asiatica.

The results of the table 4 shows there is no significant changes in body weight of various vital organs in the body in toxicity studies.

Therefore the formulation F3 is highly effective (Table 3) and it is safe (Table 4).

Note: No mortality/gross abnormality was observed in the animals during the treatment of Sesamum indicum oil containing formulation.

TABLE 5
Effect of formulation F3 in chronic stress (CS) induced perturbations
in frontal cortex of brain region and the levels of superoxide
dismutase (SOD), catalase (CAT), and lipid peroxidase (LPO)

| | Treatment | | | | |
|---|---|---|---|---|---|
| Groups | (mg/kg) | n | LPO | CAT | SOD |
| I | Normal | 10 | 2.94 ± 0.5 | 21.4 ± 2.2 | 19.3 ± 2.1 |
| II | F3 50 | 8 | 1.71 ± 0.3 | 25.2 ± 0.7^{a} | 24.6 ± 1.4 |
| III | F3 100 | 8 | 1.44 ± 0.7 | 30.1 ± 1.2^{b} | 27.1 ± 1.2^{a} |
| IV | Normal + CS | 10 | 5.62 ± 0.8 | 9.8 ± 0.9 | 39.7 ± 2.6 |
| V | F3 50 + CS | 8 | 3.91 ± 0.6 | 13.2 ± 0.8^{y} | 24.2 ± 2.9^{y} |
| VI | F3 100 + CS | 8 | 2.81 ± 0.8^{x} | 16.1 ± 0.7^{z} | 29.6 ± 2.8^{z} |

P: ^{a}<0.05 and ^{b}<0.01 compared to group I.
P: ^{x}<0.05, ^{y}<0.01 and ^{z} <0.001 compared to group IV.

Values are mean±SEM for six mice in each
Group II and Group III compare with Group I
Group V and Group VI compare with Group IV

The F3 formulation contains Sesamum indicum and Centella asiatica.

The results of table 5 represents a significant antioxidant activity by increasing the levels of catalase (CAT) and superoxide dismutase (SOD) in frontal cortex of brain region as such with F3 formulation and also in chronic stress (CS) with F3 formulation. The lipid peroxidase (LPO) product was scavenged in higher dose with F3 formulation and the levels were lowered. Therefore the F3 shows antioxidant activity in frontal cortex in brain.

Note: There is no mortality/gross abnormality was observed in the animals during the treatment of Sesamum indicum oil.

TABLE 6
Effect of formulation (F3) on chronic stress (CS) induced
perturbations in stratium of brain region and the levels of superoxide
dismutase (SOD), catalase (CAT), and lipid peroxidase (LPO).

| | Treatment | | | | |
|---|---|---|---|---|---|
| Groups | (mg/kg) | n | LPO | CAT | SOD |
| I | Normal | 10 | 3.91 ± 0.9 | 26.4 ± 1.4 | 23.2 ± 1.2 |
| II | F3 50 | 8 | 2.68 ± 0.8 | 32.2 ± 0.9 | 28.9 ± 1.4 |
| III | F3 100 | 8 | 1.95 ± 0.7** | 34.1 ± 1.2* | 34.7 ± 1.4* |
| IV | Normal + CS | 10 | 6.64 ± 0.8 | 13.8 ± 0.7 | 43.5 ± 1.7 |
| V | F₃ 50 + CS | 8 | 3.96 ± 0.9 | 17.8 ± 0.6* | 27.1 ± 0.9** |
| VI | F₃ 100 + CS | 8 | 3.78 ± 0.8* | 21.6 ± 0.9** | 21.8 ± 0.8** |

P: *<0.05 and ** <0.01 compared to Group I.
P: *<0.05 and ** <0.001 compared to Group V and Group VI.

Values are mean±SEM for six mice
Group II and Group III compare with Group I
Group V and Group VI compare with Group IV

The results showed with the F3 formulation contains Sesamum indicum and Centella asiatica a significant antioxidant activity such as with F3 formulation (Group II and III) and also with chronic stress (CS) in F3 formulation (Group V and VI) showed significant antioxidant activity by scavenging free radicals lipid peroxide (LPO) and increased the levels of catalase (CAT) and SOD (super oxide dismutase).

Note: There is no mortality/gross abnormality was observed in the animals during the treatment of Sesamum indicum oil containing formulation.

### REFERENCES CITED

1. U.S. Pat. No. 6,187,313, February, 2001, Segelman
2. U.S. Pat. No. 5,728,384, March, 1998, Tokuyama
3. Amani E. Khalifa, J Of Ethnopharmacology, 76, pp. 49-57, 2001.
4. Coyle et al, Science, 219, pp 1184-1189, 1983.
5. Eichenbaum, H., Stewart, C. and Morris, R. G., 1990. Journal of Neurosciences 10, pp. 3531-3542 1990.
6. Gibson et al J of Neuro Chemistry, 36, pp 28-33, 1981
7. Gogte. Ayurvedic Pharmacology and therapeutic use of medicinal plants. Bharatiya vidya Bhavan, Mumbia, India 2000.
8. Jinghua Xu et al, J of Ethnopharmacology, 73, pp. 405-413, 2000.
9. Mahajan et al. Int J Food Sci Nutr. 53(6) pp. 455-463, 2002.
10. Robert W. Flint, Jr, Behavioural Brain Research, 142, pp. 217-228, 2003
11. Sheila M. Mihalik, neurobiology of learning and memory, 80, pp. 55-62, 2003.
12. Singh et al J of Medicinal and Aromatic Plant sciences 22, pp 732-738, 2000.
13. So Ra Kim et al, Cognitive Brain Research, 17, pp. 454-461, 2003.
14. Tzen et al. Plant Physiol, 101(1), pp. 267-276, 1993.
15. Zaghloul and Prakash. Nahrung, 46 (5), pp. 364-369, 2002.
16. Wealth of India, pp 116-118, 1994.
17. Kirtikar & Basu. Indian medicinal plants, vol 5, pp 1658-1663.
18. Satyavati. Medicinal plants of India, vol 1, pp 216-220.
19. Veerendra kumar M H, Gupta Y K. Clin Exp Pharmacol Physiol. May-June; 30(5-6), pp 336-42.2003
20. Veerendra kumar M H, Gupta Y K. J Ethnopharmacol, February; 79(2), pp 253-260.
21. K. Nalini et al. Fitotherpia vol LXIII, No 3, pp 232-237. 1992.
22. “Charaka Samhita Chikitsa Sthana”, Ist Chapter, 3^{rd} pada, 30^{th} stanza.
23. Mukerji B, “Indian Pharmaceutical Codex”, New Delhi, India, 1953, pp 60.

1. A a synergistic herbal formulation as a brain tonic, cognition, recalling of thoughts and as an antioxiadant capable of treating or preventing amnesia and having property for improving memory, said formulation comprising pharmaceutically acceptable amounts of extracts from plants Centella asiatica and Sesamum indicum optionally along with pharmaceutically acceptable salt/s, carrier/s or dilutent/s.

2. A synergistic herbal formulation as claimed in claim 1, wherein Sesamum indicum oil is about 2 to 20% and Centella asiatica oil is about 1 to 15%.

3. A synergistic herbal formulation as claimed in claim 2, wherein Sesamum indicum oil is about 10% and Centella asiatica oil is about 5%.

4. A synergistic herbal formulation as claimed in claim 3, wherein Sesamum indicum oil is about 4% and Centella asiatica oil is about 2%.

5. A synergistic herbal formulation as claimed in claim 1, wherein the pharmaceutically acceptable salt/s, carrier/s or dilutent/s are selected from group comprising of lactose, mannitol, sorbitol, microcrystalline cellulose, sucrose, sodium citrate, sodium chloride or dicalcium phosphate.

6. A synergistic herbal formulation as claimed in claim 1, wherein said formulation has a high antioxidant, cooling, oleaginous, diuretic and nerve relaxant properties.

7. A synergistic herbal formulation as claimed in claim 1, wherein the said formulation may be delivered in the form of capsule, tablet, syrup, suspension, pills or elixirs.

8. A synergistic herbal formulation as claimed in claim 1, wherein extract of the formulation is obtained from leaves of Centella asiatica and seeds of Sesaumum indicum.

9. A synergistic herbal formulation as claimed in claim 1, wherein plant parts are selected from a group consisting of seeds of white and black varieties and leaves.

10. A synergistic herbal formulation as claimed in claim 1, wherein said formulation is useful for curing migraine, vertigo, leucoderma, anaemia and improve appetite.

11. A synergistic herbal formulation as claimed in claim 1, wherein said formulation may be used for curing wounds, fractures, syphilitic skin diseases, both externally and internally and also in treatment of leprosy and to ameliorate the symptoms of disease and to improve the general health of the patient.

12. A synergistic herbal formulation as claimed in claim 1, wherein saiod formulation is useful in reducing reduce the pain of piles, stomachic, and enlargement of spleen.

13. A synergistic herbal formulation as claimed in claim 1, wherein dosage of the formulation in the range of about 20-110 mg/kg does not show abnormality of the locomotor activity, on passive avoidance test showed significant and dose dependent activity, showed significant and dose dependent antioxidant activity of the frontal cortex and of striatum regions of the brain.

14. A synergistic herbal formulation as claimed in claim 13, wherein dosage of the said formulation in the range of about 25-100 mg/kg does not show abnormality of the locomotor activity, on passive avoidance test shows significant and dose dependent activity and shows significant and dose dependent antioxidant activity of the frontal cortex and of striatum regions of the brain.

15. A synergistic herbal formulation as claimed in claim 1, wherein dosage of synergistic formulation reduces the impairment of memory acquisition by reducing the latency period in the range of about 0.05 to 2.0 seconds.

16. A synergistic herbal formulation as claimed in claim 15, wherein dosage of synergistic formulation reduces the impairment of memory acquisition by reducing the latency period in the range of about 0.18 to 1.22 seconds.

17. A synergistic herbal formulation as claimed in claim 1, wherein dosage of synergistic formulation reduces the impairment of memory acquisition by reducing number of mistakes in the range of about 1 to 35.

18. A synergistic herbal formulation as claimed in claim 17, wherein dosage of synergistic formulation reduces the impairment of memory acquisition by reducing the number of mistakes in the range of about 6.1 to 27.

19. A synergistic herbal formulation as claimed in claim 1, wherein dosage of synergistic formulation enhances the body weight in the range of about 140 to 170 gms.

20. A synergistic herbal formulation as claimed in claim 19, wherein dosage of synergistic formulation enhances the body weight in the range of about 141.6 to 168.7 gms

21. A synergistic herbal formulation as claimed in claim 1, wherein dosage of synergistic formulation enhances the kidney weight in the range of about 0.8 to 1.5 gms.

22. A synergistic herbal formulation as claimed in claim 21, wherein dosage of synergistic formulation enhances the kidney weight in the range of about 0.82 to 1.03 gms.

23. A synergistic herbal formulation as claimed in claim 1, wherein dosage of synergistic formulation enhances the liver weight in the range of about 4 to 7 gms.

24. A synergistic herbal formulation as claimed in claim 23, wherein dosage of synergistic formulation enhances the liver weight in the range of about 5.26 to 6.42 gms

25. A synergistic herbal formulation as claimed in claim 1, wherein dosage of synergistic formulation enhances the spleen weight in the range of about 0.60 to 0.80 gms.

26. A synergistic herbal formulation as claimed in claim 25, wherein dosage of synergistic formulation enhances the spleen weight in the range of about 0.63 to 0.76 gms.

27. A synergistic herbal formulation as claimed in claim 1, wherein dosage of synergistic formulation under non-stress conditions lowers the lipid peroxidase (LPO) activity in the frontal cortex and stratium regions of the brain in the range of 1.0 to 5.0.

28. A synergistic herbal formulation as claimed in claim 27, wherein dosage of synergistic formulation under non-stress conditions lower the lipid peroxidase (LPO) activity in the frontal cortex and stratium regions of the brain in the range of 0.74 to 3.48.

29. A synergistic herbal formulation as claimed in claim 1, wherein dosage of synergistic formulation under non-stress conditions enhances the catalase (CAT) activity in the frontal cortex and stratium regions of the brain in the range of 22 to 40.

30. A synergistic herbal formulation as claimed in claim 29, wherein dosage of synergistic formulation under non-stress conditions enhances the catalase (CAT) activity in the frontal cortex and stratium regions of the brain in the range of 24.5 to 35.3.

31. A synergistic herbal formulation as claimed in claim 1, wherein dosage of synergistic formulation under non-stress conditions enhances the superoxide dismutase (SOD) in the frontal cortex and stratium regions of the brain activity in the range of 22 to 40.

32. A synergistic herbal formulation as claimed in claim 31, wherein dosage of synergistic formulation non-stress conditions enhance the superoxide dismutase (SOD) activity in the frontal cortex and stratium regions of the brain in the range of 23.2. to 30.3.

33. A synergistic herbal formulation as claimed in claim 1, wherein dosage of synergistic formulation under stress conditions lower the LPO activity in the frontal cortex and stratium regions of the brain in the range of about 1 to 7.

34. A synergistic herbal formulation as claimed in claim 33, wherein dosage of synergistic formulation under stress conditions lower the LPO activity in the range of about 2.8 to 4.86.

35. A synergistic herbal formulation as claimed in claim 1, wherein dosage of synergistic formulation under chronic stress conditions enhance CAT activity in the frontal cortex and stratium regions of the brain in the range of 10 to 25.

36. A synergistic herbal formulation as claimed in claim 35, wherein dosage of synergistic formulation under chronic stress conditions enhance CAT activity in the frontal cortex and stratium regions of the brain in the range of 12.4 to 22.5.

37. A synergistic herbal formulation as claimed in claim 1, wherein dosage of synergistic formulation under chronic stress conditions lower the SOD activity in the frontal cortex and stratium regions of the brain in the range of 20 to 35.

38. A synergistic herbal formulation as claimed in claim 37, wherein dosage of synergistic formulation under chronic stress conditions lower SOD activity in the frontal cortex and stratium regions of the brain in the range of 21 to 33.

39. A method of preparing a synergistic herbal formulation as a brain tonic, cognition, recalling of thoughts and as an antioxiadant capable of treating or preventing amnesia and having property for improving memory, said method comprising steps of:
a. extracting the powdered material obtained from seeds of Sesamum indicum and leaves of Centella asiatica in aqueous alcohol,
b. filtering the extract of step (a) to remove the debris,
c. concentrating and lyophislizing the filtrate obtained from step (b) at a temperature of less than about 55° C., and
d. mixing the plant extracts obtained in step (c) with carbohydrates of about 70% and alcohol of about 12% to make a volume of 100 ml to obtain the formulation

40. A method as claimed in claim 39, wherein aqueous alcohol in the step (a) is about 60%.

41. A method as claimed in claim 40, wherein aqueous alcohol in the step (a) is about 50%.

42. A method as claimed in claim 39, wherein aqueous alcohol in step (a) is ethanol.

43. A method as claimed in claim 39, wherein temperature in the step (b) is about 50° C.

44. A method as claimed in claim 39, wherein carbohydrates in step (d) are selected from sucrose or lactose.

45. A method as claimed in claim 39, wherein carbohydrate concentration is about 66%.

46. A method as claimed in claim 39, wherein alcohol in step (d) is about 10%.

47. A method as claimed in claim 39, wherein Sesamum indicum oil is in the range of about 2 to 20% and Centella asiatica oil is in the range of about 1 to 15%.

48. A method as claimed in claim 47, wherein Sesamum indicum oil is about 10% and Centella asiatica oil is about 5%.

49. A method as claimed in claim 48, wherein Sesamum indicum oil is about 4% and Centella asiatica oil is about 2%.

50. A method as claimed in claim 39, wherein synergistic formulation has a high antioxidant, cooling, oleaginous, diuretic and nerve relaxant properties.

51. A method as claimed in claim 39, wherein synergistic formulation may be delivered in form of capsule, tablet, syrup, suspension, pills or elixirs.

52. A method as claimed in claims 39, wherein plant parts are selected from a group consisting of seeds of white and black varieties and leaves.

53. A method treating and/or preventing amnesia and improving memory in a mammal, particulary humans said method comprsing administering synergistic herbal formulation of extracts from plants Centella asiatica and Seasmum indicum optionally along with pharamaceutically acceptable salt/s, carrier/s or dilutent/s to a subject.

54. A method as claimed in claim 53, wherein synergistic formulation is useful for curing migraine, vertigo, leucoderma, anaemia and improve appetite.

55. A method as claimed in claim 53, wherein synergistic formulation is useful for curing wounds, fractures, syphilitic skin diseases, both externally and internally and also in treatment of leprosy and to ameliorate the symptoms of disease and to improve the general health of the patient.

56. A method as claimed in claim 53, wherein synergistic formulation is useful in reducing reduce the pain of piles, stomachic, and enlargement of spleen.

57. A method as claimed in claim 53, wherein pharmaceutically acceptable salt/s, dilutent/s, carrier/s are selected from group comprising of lactose, mannitol, sorbitol, microcrystalline cellulose, sucrose, sodium citrate, sodium chloride or dicalcium phosphate.

58. A method as claimed in claim 53, wherein dosage of synergistic formulation in the range of about 20-110 mg/kg does not show abnormality of the locomotor activity, on passive avoidance test showed significant and dose dependent activity, showed significant and dose dependent antioxidant activity of the frontal cortex and of striatum regions of the brain.

59. A method as claimed in claim 58, wherein dosage of synergistic formulation in the range of about 25-100 mg/kg does not show abnormality of the locomotor activity, on passive avoidance test shows significant and dose dependent activity and shows significant and dose dependent antioxidant activity of the frontal cortex and of striatum regions of the brain.

60. A method as claimed in claim 53, wherein dosage of synergistic formulation reduces the impairment of memory acquisition by reducing the latency period in the range of about 0.05 to 2.0 seconds.

61. A method as claimed in claim 60, wherein dosage of synergistic formulation reduces the impairment of memory acquisition by reducing the latency period in the range of about 0.18 to 1.22 seconds.

62. A method as claimed in claim 53, wherein dosage of synergistic formulation reduces the impairment of memory acquisition by reducing number of mistakes in the range of about 1 to 35.

63. A method as claimed in claim 62, wherein dosage of synergistic formulation reduces the impairment of memory acquisition by reducing the number of mistakes in the range of about 6.1 to 27.

64. A method as claimed in claim 53, wherein dosage of synergistic formulation enhances the body weight in the range of about 140 to 170 gms.

65. A method as claimed in claim 64, wherein dosage of synergistic formulation enhances the body weight in the range of about 141.6 to 168.7 gms

66. A method as claimed in claim 53, wherein dosage of synergistic formulation enhances the kidney weight in the range of about 0.8 to 1.5 gms.

67. A method as claimed in claim 66, wherein dosage of synergistic formulation enhances the kidney weight in the range of about 0.82 to 1.03 gms.

68. A method as claimed in claim 53, wherein dosage of synergistic formulation enhances the liver weight in the range of about 4 to 7 gms.

69. A method as claimed in claim 68, wherein dosage of synergistic formulation enhances the liver weight in the range of about 5.26 to 6.42 gms.

70. A method as claimed in claim 53, wherein dosage of synergistic formulation enhances the spleen weight in the range of about 0.60 to 0.80 gms.

71. A method as claimed in claim 70, wherein dosage of synergistic formulation enhances the spleen weight in the range of about 0.63 to 0.76 gms.

72. A method as claimed in claim 53, wherein dosage of synergistic formulation under non-stress conditions lowers the lipid peroxidase (LPO) activity in the frontal cortex and stratium regions of the brain in the range of 1.0 to 5.0.

73. A method as claimed in claim 72, wherein dosage of synergistic formulation under non-stress conditions lower the lipid peroxidase (LPO) activity in the frontal cortex and stratium regions of the brain in the range of 0.74 to 3.48.

74. A method as claimed in claim 53, wherein dosage of synergistic formulation under non-stress conditions enhances the catalase (CAT) activity in the frontal cortex and stratium regions of the brain in the range of 22 to 40.

75. A method as claimed in claim 74, wherein dosage of synergistic formulation under non-stress conditions enhances the catalase (CAT) activity in the frontal cortex and stratium regions of the brain in the range of 24.5 to 35.3.

76. A method as claimed in claim 53, wherein dosage of synergistic formulation under non-stress conditions enhances the superoxide dismutase (SOD) in the frontal cortex and stratium regions of the brain activity in the range of 22 to 40.

77. A method as claimed in claim 76, wherein dosage of synergistic formulation non-stress conditions enhance the superoxide dismutase (SOD) activity in the frontal cortex and stratium regions of the brain in the range of 23.2. to 30.3

78. A method as claimed in claim 53, wherein dosage of synergistic formulation under stress conditions lower the LPO activity in the frontal cortex and stratium regions of the brain in the range of about 1 to 7.

79. A method as claimed in claim 78, wherein dosage of synergistic formulation under stress conditions lower the LPO activity in the range of about 2.8 to 4.86.

80. A method as claimed in claim 53, wherein dosage of synergistic formulation under chronic stress conditions enhance CAT activity in the frontal cortex and stratium regions of the brain in the range of 10 to 25.

81. A method as claimed in claim 80, wherein dosage of synergistic formulation under chronic stress conditions enhance CAT activity in the frontal cortex and stratium regions of the brain in the range of 12.4 to 22.5.

82. A method as claimed in claim 53, wherein dosage of synergistic formulation under chronic stress conditions lower the SOD activity in the frontal cortex and stratium regions of the brain in the range of 20 to 35.

83. A method as claimed in claim 82, wherein dosage of synergistic formulation under chronic stress conditions lower SOD activity in the frontal cortex and stratium regions of the brain in the range of 21 to 33.
